(19) United States
(12) Patent Application Publication
(10) Pub. No.: US 2009/0068715 A1
(43) Pub. Date: Mar. 12, 2009

(54) METHOD OF PRODUCING BIO-ETHANOL

(75) Inventors: Masahiro Ogaki, Kawachinagano (JP); Shuzo (John) Tanaka, Stamford, CT (US); Haruhiko Kawasaki, Sakai (JP); Takasada Ishii, Sakai (JP)

Correspondence Address: DOBRUSIN & THENNISCH PC, 29 W LAWRENCE ST, SUITE 210, PONTIAC, MI, 48342, US

(73) Assignees: OGAKI Bio. Technology Research Co., Ltd.; LOVION INTERNATIONAL, INC., Stamford, CT (US); OSAKA PREFECTURE UNIVERSITY, Algae Bio-Engineering Research Laboratory

(21) Appl. No.: 12/194,823

(22) Filed: Aug. 20, 2008

Related U.S. Application Data
(60) Provisional application No. 60/970,274, filed on Sep. 6, 2007.

(51) Int. Cl. C12P 7/08; C12P 7/06

(52) U.S. Cl. 435/163; 435/161

(57) ABSTRACT

A method of producing ethanol, which comprises of starch obtained from continuously cultivated unicellular green algae strains which reproduce through a single cell clone cultivation method that cyclically produces starch extra-cellularly. Only the starch is recovered and goes through a saccharification and fermentation process for the production of ethanol. The algae are left for continual reprocessing. The obtained starch is then saccharified and fermented to produce ethanol. This production method is available and feasible in any part the world, from tropical areas to high latitude areas because it is controlled not by natural climatic conditions but in an environment that is monitored and controlled by humans. The continuous production process from these Chlorella algae strains can reduce the production cost of ethanol production as well as contribute to reducing industrial wastes and carbon dioxide to contribute to the earth's environmental wellbeing.

### CLAIM OF BENEFIT OF FILING DATE

The present application claims the benefit of the filing date of U.S. Provisional Application Ser. No. 60/970,274 (filed Sep. 6, 2007), hereby incorporated by reference.

### FIELD OF THE INVENTION

This invention concerns an improved method of producing ethanol from unicellular green algae, more particularly an improved method of producing ethanol from a starch obtained from continuously cultivated unicellular green algae strains which reproduce through a single cell cloning cultivation method that cyclically produces starch extra-cellularly. Only the starch is recovered and goes through a saccharification and fermentation process for the production of ethanol. The algae are left for continual reprocessing.

### BACKGROUND OF THE INVENTION

There have been concerns over the last century about the expected shortage of our natural resources such as fossilized petroleum as well as increasing environmental contamination such as air pollution, particularly by carbon dioxide, that will inevitably affect us all in the near future.

It is known by scientists in the field that ethanol can be obtained by fermentation of the starch produced by higher plants such as corn, wheat and sweet potatoes. However, the growth of these plants is influenced by the weather and their yields depend, to a large extent, upon climatic conditions. Furthermore, with current threats of food shortages and increased food costs throughout the world, it is likely to become difficult to obtain ethanol from such agricultural products.

A new ethanol production process that avoids the use of weather controlled plant life is desired. Using unicellular green algae strains to produce starch effectively reduces the need to rely upon unpredictable plant life for starch. This obtained starch can be fermented to produce bio-ethanol.

The present invention is based on the use of a unicellular green algae with the disclosed improved processing method, particularly the use of a heterotrophic chlorella strain Chlorella vulgaris Al-1y and its related strains in conjunction with the improved method. This chlorella consumes CO₂ as one of its nutrients in its production process. This consumption of CO₂ could contribute to the earth's environmental well-being.

Microbial materials that are obtained through fermentation technology have been widely used in both food and medical products and a variety of additives and enzymes that are obtained from these microbial materials are utilized in a number of industrial fields. However, the material obtained specifically from microbial algae has not been used as widely. Presently, there are not many uses of unicellular green algae strains except for use in some polysaccharides such as agar, alginic acid, and karaginin which are used as viscosity and gel enhancers. The research fields of unicellular algae strains have also not been developed due to the slow cultivation speed as compared with other microbial materials.

A variety of algae strains exist in fresh water, seawater and other environments such as hot springs. These algae have a wide array of biological characteristics that allow them to adapt to their specific environment. There are autotrophic algae that require light for growth and heterotrophic species that require no light for growth. Both types are used a microbiological materials. One aim of the present invention is to identify and utilize an algae that is capable of assimilating carbon dioxide and other types of industrial waste so as to contribute to the earth's environmental well-being while also producing starch effectively.

It is known that a specific algae strain of Chlorella vulgaris Al-1y and its related strains can assimilate carbon dioxide and other organic industrial by-products while producing substantial quantities of starch either intra-cellularly or extra-cellularly. The starch production of these strains is a unique characteristic according to Japanese Patent Application No. 52-082793 and Japanese Patent No. 1023001. The methods of producing ethanol from the starch produced by chlorella are stated in Japanese Patent Application Nos. 50-148587, 54-011397, Japanese Patent No. 0979722, Great Britain Patent No. 1 493 480 and U.S. Pat. No. 5,578,472. Ethanol production methods using seawater algae are disclosed in Japanese Patent Application Nos. 2000-316593 and 2003-310228, and Japanese Patent No. 3866144. These algae also assimilate ashes as inorganic nutrients.

The fermentation process of the prior art results in destruction of the whole single cell clone cultivations during removal of the starch from the algae. The present invention avoids this outcome by using a continuous cultivation method that does not destroy the algae cells during starch removal.

### SUMMARY OF THE INVENTION

In a first aspect, the present invention contemplates a method for producing ethanol comprising cultivating starch producing unicellular green algae within a sealed tank, wherein the algae reproduce through a single cell cloning cultivation that cyclically produces starch extra cellularly, recovering the starch, wherein the algae remain in the tank for continued cultivation by adding additional Vulgaris Al-1y seed material, saccharifying the starch, fermenting the saccharified starch to produce a fermentation medium, producing ethanol from the fermented and saccharified starch, and recovering the ethanol produced from the fermentation medium.

This aspect may be further characterized by one or any combination of the following features: the algae used are strains of Chlorella vulgaris and its induced strains, the algae are cultured at a temperature in the range of 25-42° C. and at a pH in the range of 5-9 in a closed tank, the algae are exposed to radiant ray or ultraviolet ray irradiation to produce more productive and selectable starch rich chlorella, the Chlorella vulgaris and its induced strains are cultivated under autotrophic conditions, the Chlorella vulgaris and its induced strains are cultivated under heterotrophic or mixotrophic conditions, wherein an aseptic sealed tank contains an assimilable nutritional cultivating medium, the assimilable nutritional cultivating medium contains acetic acid or organic acid wastes or by-products, the assimilable nutritional cultivating medium contains carbon dioxide gas or carbonic acid salts, prior to fermentation the saccharified starch is exposed to microwave irradiation within a sealed pipeline, so that the starches are degenerated, the cultivation medium includes industrial waste, by-products, or combinations thereof.

In another aspect, the present invention contemplates a method for producing ethanol comprising cultivating Chlorella vulgaris under mixotrophic conditions within an aseptic sealed tank containing acetic acid, organic acid, carbon dioxide gas, carbonic acid salts or combinations thereof, at a temperature in the range of 25-42° C. and at a pH in the range of 5-9, wherein the algae reproduce through a single cell cloning cultivation that cyclically produces starch extra cellularly, recovering the starch, wherein the algae remain in the tank for continued cultivation by adding additional Vulgaris Al-1y seed material, saccharifying the starch, exposing the saccharified starch to microwave irradiation within a sealed pipeline, so that the starches are degenerated, fermenting the saccharified starch to produce a fermentation medium, producing ethanol from the fermented and saccharified starch, and recovering the ethanol produced from the fermentation medium.

This aspect may be further characterized by the cultivation medium including industrial waste, by-products or combinations thereof.

### BRIEF DESCRIPTION OF THE DRAWINGS

FIG. 1 is a flow chart depicting the bio-ethanol production process as taught in the present invention.

FIG. 2 is the Chlorella vulgaris cultivation growth speed curve as measured by spectrophotometer, the volume of the Chlorella vulgaris strain in a 1.5 liter fermentation tank, and the number of hours of cultivation.

FIG. 3 is the Chlorella vulgaris cultivation growth speed curve as measured by a blood cell counter, the volume of the Chlorella vulgaris strain in a 1.5 liter fermentation tank, and the number of hours of cultivation.

### DETAILED DESCRIPTION

The present invention is based upon the use of a unicellular green algae to produce bio-ethanol with the disclosed improved processing method, particularly the use of a heterotrophic chlorella strain Chlorella vulgaris Al-1y and its related strains in conjunction with the improved method.

These particular Chlorella vulgaris strains do not require a strong light source in their photosynthesis processes for producing chlorophyll. These particular strains contain starch intra and extra-cellularly and can be cultivated in a sealed tank which means that it has both autotrophic and heterotrophic cultivation possibilities. The present invention allows for only the starch to be recovered from the cultivation tank, while the algae remain in the tank for continual reprocessing by adding additional Vulgaris Al-1y seed material. Up until now, the fermentation process by regular technology, during the separation process of the starch from the algae, whole single cell clone cultivations are destroyed and wasted and the process is never continuously done. Mass production of the ethanol was never achieved with this process because it proved to be too expensive. From this continuous production of starch induced from the algae, it can efficiently be made ready for bio-ethanol production through saccharification, the sealed pipeline and fermentation.

The present invention includes a cultivation method in a completely sealed tank using these Chlorella vulgaris strains that results in high starch-producing Chlorella. The resulting starch can be further processed into bio-ethanol. Such a process would not be influenced by weather or any geological conditions so that the bio-ethanol can be produced all year long under human control. The cultivation method of the present invention can take place in any extreme climate including areas of low sunlight, areas of ultra low or ultra high temperatures and areas of high altitude. In one preferred embodiment, the production of bio-ethanol according to the present invention may produce up to ten times as much ethanol as traditional production using corn or sugarcane.

Use of this improved method of producing ethanol with Chlorella vulgaris Al-1y micro algae and its induced strains has shown to be more economical, higher in productivity, and a simplified process of bio-ethanol production than previously tried methods.

In one preferred embodiment, starch makes up from about 50 to 90% of the dry weight of the algae in strains such as Chlorella vulgaris Al-1y. More preferably, starch makes up from about 70 to 80% of the Chlorella vulgaris Al-1y strain. Further, the strains have autotrophic and heterotrophic photosynthetic properties.

In a preferred embodiment, these strains can be cultured in a sealed dark tank or a tank with windows allowing for light. These strains are thus considered “mixotrophic” meaning that the Chlorella vulgaris Al-1y and similar strains do not require light for photosynthesis, but rather they have the option of using light during cultivation. Additionally, these strains have a defective alpha cell wall outside of each cell. This means that the algae are easier to digest and that the starch extraction for fermentation process is simplified and less invasive because there is starch outside the cell wall and the starch from within the cell wall often seeps out from the cell wall. Further, the starch may be collected without complete destruction of the algal cells. The mixotrophic culture for the Chlorella vulgaris Al-1y and related chlorella strains has a unique characteristic in that it can be cultivated in a completely sealed tank. This mixotrophic cultivation characteristic means that these have both autotrophic and heterotrophic cultivation possibilities. This characteristic is found only in the Al-1y strains. These starch rich Al-1y and its related chlorella strains can be cultivated in a sterilized germ free tank with little or no light in human controlled conditions as opposed to an open air tank that can be exposed to many outside factors that cannot be controlled. The mixotrophic tank cultivation is far different from a conventional chlorella production, i.e. a regular autotrophic chlorella being cultured in an open pond. This mixotrophic cultivation is done in a germ free tank that can be sterilized and controlled, providing the nutritional supply condition more economically, particularly in the consumption of CO₂ and other nutrients such as glucose, acetic acid, saccharides, lipids, organic acid or industrial by-products and wastes. With chlorella cultured in an open tank, certain factors cannot be controlled, such as temperature, germ free conditions, climate etc. Further, the consumption of carbon dioxide and industrial by-products and wastes prevents environmental pollution. This process can be carried out under any severe environmental circumstances such as low sunlight, low average temperature, at any latitude, wherever we can use industrial by-products, organic wastes, exhausted carbon dioxide and light concentration devices such as glass fibers because the algae is highly adaptable. The surplus energy from these wastes and byproducts can also be used.

In most of these chlorella strains, the cells divide up to a maximum of about 32 cells in each continuous about 72 hour period under specialized conditions. The resulting mixotrophic cultivation will have more economic potential compared to the cultivation of regular chlorella, which is cultivated in an open pond and usually takes several months.

Other strains of algae have potential for use in ethanol production, as in Example 1, such as Chlamydomonas sp. Al-5, Scenedesmus basilensis, Scenedesmus basilensis IAM C-66 and others.

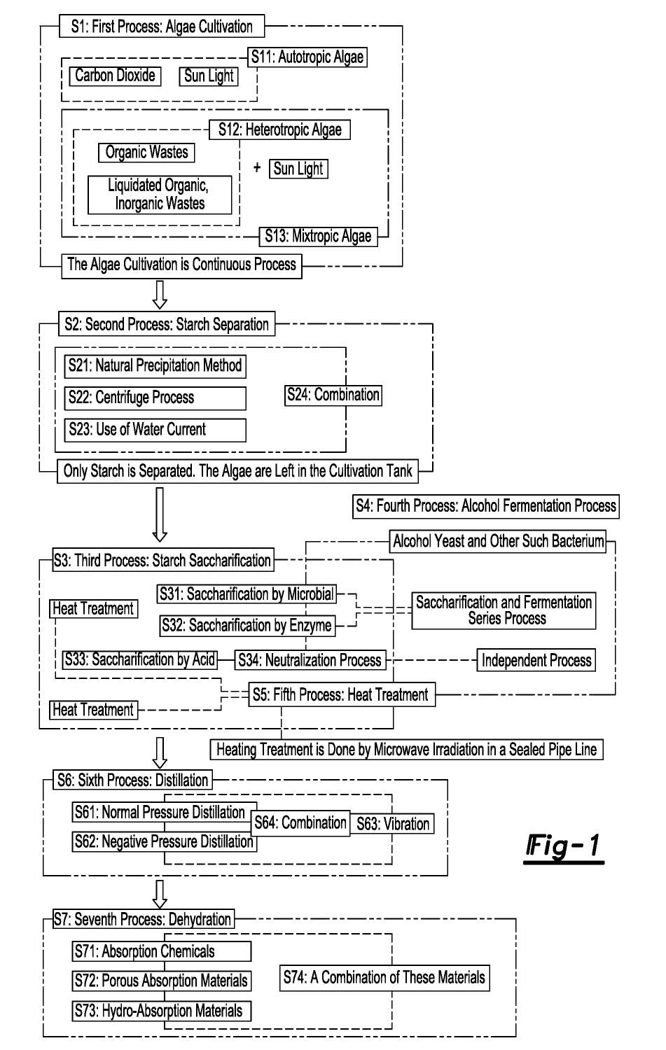

During the continuous cultivation process of unicellular green algae strains, the starch that is produced extra-cellularly is easy to recover. The algae can therefore be left intact to reproduce in a single cell cloning cultivation method for continual reprocessing by adding additional Vulgaris Al-1y seed material. Therefore, the present invention allows for continual production of bio-ethanol as shown in FIG. 1. In a preferred embodiment, the production of bio-ethanol occurs in seven processing steps. S1: algae cultivation; S2: starch separation; S3: starch saccharification; S4: alcohol fermentation; S5: heating; S6: distillation and S7: dehydration.

In a preferred embodiment, S1 (cultivation) includes three different manners of cultivation: autotrophic (S11), heterotrophic (S12), and mixotrophic (S13). The autotrophic cultivation (S11) uses a standard photosynthetic process, requiring inorganic salts and carbon dioxide as nutrition for photosynthesis. The S11 process cannot proceed without light. The heterotrophic cultivation (S12) uses no light but requires additional organic material for nutrition. S13, the mixotrophic cultivation, is a combination of both S11 (autotrophic), and S12 (heterotrophic) processes. S2 (starch separation) includes several separation processes: S21 (decantation by natural precipitation); S22 (separation by centrifugal force); and S23 (separation by using slow water current in a tank). S21 does not require additional apparatus, but takes more time and requires a large scale container. S22 is the most common separation system, but does not separate as thoroughly as S21. S23 is a good fit for continual large volume separation. As shown in FIG. 1, S24 is a combination of these processes. The S3 (saccharification) process is also selectable from a number of different saccharification methods. S31 uses a microbial, S32 uses an enzyme, S33 uses an acid followed by neutralization (S34) by adding an additional acid or alkaline solution such as sulfuric acid or sodium hydroxide.

The S4 (alcohol fermentation) process uses alcohol yeast, alcohol yeast bacterium and other microbacteria. The S5 (distillation or heating) process causes starch hydrolysis and uses microwave irradiation through a sealed production pipeline. The microwave length used is that of conventional waves such as about 2.45 gigahertz and about 9.15 megahertz.

The S6 (distillation) process includes three types of distillation processes including distillation under normal pressure (S61), distillation under negative pressure (S62), and distillation stimulated by ultrasound vibrations (S63). In one preferred embodiment, the present invention uses a combination of these processes (S64). There are also a number of different S7 (dehydration) processes that may be used. S71 uses anhydrous chemical agents such as sodium sulfate anhydride or a metal oxide such as lime. S72 uses dehydration agents of high porosity materials such as zeolite. S73 uses hydro-absorption agents such as silica gel. In one preferred embodiment, the present invention uses a combination of these processes (S74).

In another preferred embodiment, the present invention includes the use of radiant ray irradiation or another mutation inducer for producing proper and continuous mutations for more productive strains and for better selection of the highly useful, starch rich chlorella. In another preferred embodiment, the use of radiant ray irradiation such as gamma ray or ultraviolet ray on the Chlorella vulgaris Al-1y and its induced strains results in better production efficiency and a better selection method of the more useful starch rich chlorella material. The use of repeated radiant ray irradiation or other mutation inducers result in more ideal and selectable Chlorella vulgaris.

In one preferred embodiment, the Chlorella vulgaris Al-1y strains are easily identified from a culture colony by their light yellowish green color, simplifying the selection and isolation of the Chlorella vulgaris Al-1y. A cultivation medium must be devised to provide the algae with assimilable nutrients. Traditional autotrophic algae usually require a nitrogen source within the cultivation medium such as nitric acid or ammonia. Phosphoric acid and/or magnesium may be added as well in addition to small amounts of calcium, iron or other metals. Traditional heterotrophic algae require a similar cultivation medium with the addition of certain organic materials. In the cultivation of both autotrophic and heterotrophic algae, carbon dioxide and air are both blown into the cultivation medium.

In a preferred embodiment, a combination of the required nutrients for both autotrophic and heterotrophic algae is provided for the mixotrophic algae of the present invention. Preferably, the cultivation medium undergoes light irradiation during addition of the carbon dioxide and air.

In another preferred embodiment, the production process of the present invention is simplified by utilizing microwave irradiation on the recovered starch after the tank cultivation process. By using a microwave irradiation process on the recovered starch in a sealed pipe type process, the present invention eliminates a number of difficulties often encountered in the traditional processes, including the use of large amounts of energy and time. Preferably, electro magnetic microwaves are used in the range of 100 μm to 1 m and 300 megahertz to 3 terahertz.

Traditional cultivation processes require a centrifugal separation after the cultivation process and the finished products require additional processes of a heating treatment at about 100° C. for about 3 minutes. This process is required for diminishing the chlorophyllase activity and eliminating the PP (pheophorbide, a toxic substance) produced during chlorella cultivation. An additional cooling and spray drying process is also required. A very fine dry chlorella is obtained from these steps. This traditional process usually requires expensive equipment and consumes a great deal of electrical power.

In one preferred embodiment, the production method of the present invention uses a sealed pipe type processing line for the recovered starch. This process eliminates the steps described above by reduced time and energy expended and subsequently reduces the cost associated with the traditional process described above.

In one preferred embodiment, the starch obtained from the selected Chlorella Al-1y is collected according to the selection process shown in Example 1. The collected starch then undergoes a saccharification process. The starch is exposed to a final fermentation process for ethanol production, or a combination process with the microwave irradiation line. Saccharification of the starch and subsequent fermentation of the saccharified product to produce ethanol are by conventional techniques. These processes are illustrated in FIG. 1.

In one preferred embodiment, saccharification may conveniently be achieved by adding to the starch containing medium, an aqueous sulfuric acid (water to sulfuric acid weight ratio about 2:1 to 5:1) in an amount of about 5-25% by weight of sulfuric acid with respect to the quantity of the starch to be treated. Preferably, the acidified medium is then heated on a water bath (at about 100° C.) for about thirty minutes. The acid solution is then diluted with a two to five fold quantity of water and the saccharifying operation is completed by heating to about 120° C. under pressure (about 2 kg/cm2) for about thirty minutes. The saccharified liquor thus obtained is then neutralized to a pH value of about 5.0 by adding milk of lime and after filtration MgSO₄.7H₂O, KH₂PO₄ and urea are added to form a suitable fermentation medium. In another preferred embodiment, another alkaline material or industrial ash waste may be used to control the pH of the process. The neutralized medium is then inoculated with about 10% by weight of the ethanol producing yeast and the liquor remains as is for about 3 or 4 days to ferment. At the end of the fermentation process the broth contains ethanol (about 6%) which can be recovered by distillation.

In another preferred embodiment, an alternative saccharification process is used where dilute hydrochloric acid is added to the medium, which contains the starch and is then heated at about 120° C. under pressure (about 2 kg/cm2) for about thirty minutes. Subsequently the liquid is saccharified with the addition of suitable saccharified liquor, which can then be fermented as described above. The method of this invention is illustrated in the following examples referring to FIG. 3 and Table 1.

The following examples demonstrate the use of the processes discussed above and show a correlation between the volume of obtained starch and the yield of bio-ethanol.

### EXAMPLE 1

About 1 liter of a basic cultivation medium is prepared with about 2 g of NaNO₃, about 0.2 g of MgSO₄.7H₂O, about 0.05 g of CaCl₂.2H₂O, about 0.8 g of K₂HPO₄ about 0.25 g of KH₂PO₄, about 0.25 g of FeSO₄.7H₂O, about 3 mg of H₃BO₃, about 2 mg of MnCl₂.4H₂O, about 500 μg of Co(NO₃)₂.6H₂O, about 20 μg of ZnSO₄.4H₂O, about 8 μg of CuSO₄.5H₂O, and about 2 μg of Na₂MoO4.2H₂O dissolved in water. As a preservation medium, 15 g of agar-agar and 15 g of glucose are added to the basic cultivation medium. Heat is then applied to thoroughly dry out the controlled medium and the resulting dry medium is placed on a slant agar-agar preservation plate as a dry-bed medium. An additional cultivation medium for the algae is prepared using 20 g of ammonium acetate, 8 g of sodium acetate, and 7 g of potassium acetate for additional carbon nutrients. This medium is combined with the basic cultivation medium so that heterotrophic cultivation with no light source can commence. The pH of the cultivation medium needs to be monitored and controlled so that the pH remains between 6.5 and 7.5. The pH is controlled by adding acetic acid, as the pH of the cultivation medium tends to become increasingly basic during cultivation. The cultivation medium is sterilized by vapor at about 121° C. for about 15 minutes.

The Chlorella vulgaris Al-1y strains from the slant preservation medium are seeded in 10 ml of the cultivation medium described above in 100 ml triangle flasks topped with an air permeable silicon cap. The flasks are then shaken for 72 hours at 35° C. by a rotary shaker (110 shakes/minute).

The liquid is then transferred into 90 ml of a the cultivation medium in a 500 ml flask, totaling 100 ml in liquid and cultivation is continued for 40 hours under the same conditions described above. This liquid is transferred again into a 1.5 liter fermentation tank with another 900 ml of the above-mentioned cultivation medium totaling 1 liter in liquid and is supplied with air as small bubbles in the same volume as the cultivation solution every minute and is stirred at a speed of 100 rpm for 96 hours. Examination of the algae cultivation speed is measured through a light absorption method by spectrophotometer at a 540 nm wavelength and also a blood cell counter every 8 hours. The processing proceeds until just before the algae reach the conclusion of the cultivation increase logarithm. After cultivation is finished, all the algae cells are separated by a centrifuge for 10 minutes at 2,000 (G) and dried by a heating process at 105° C. The total sugar volume inside the algae cells is calculated by using the Anthrone-sulfuric acid method. The reduced sugar volume is then deducted by the Nelson-Somogyi method which is then multiplied by 0.9.

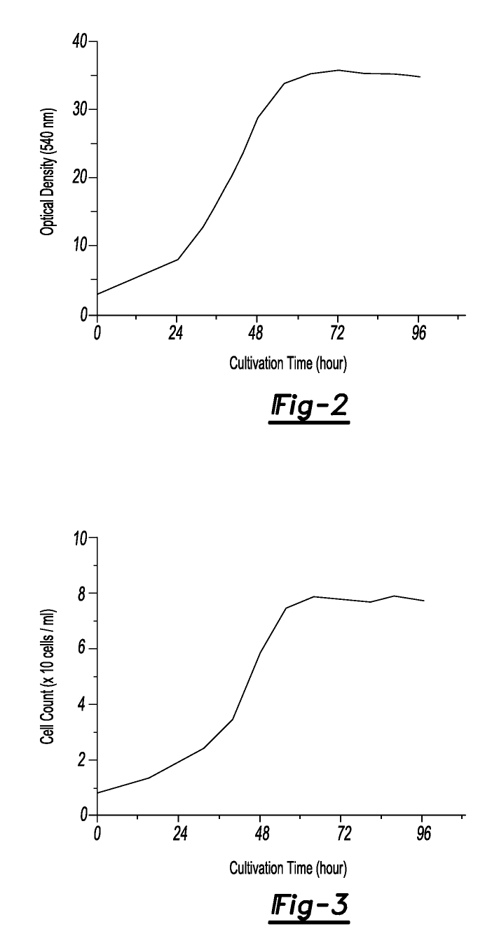

As shown in FIG. 2 and FIG. 3, after 56 hours of cultivation, the volume of the cultivated chlorella can reach the maximum volume and after 96 hours, the volume of dried algae in 1 liter of cultivation liquid was 7.5 g and the volume of starch was 5.6 g. The starch produced was about 75% of the weight volume of the algae cell. These results demonstrate the increase in starch recovery from the Chlorella vulgaris Al-1y strain, which produce starch internally and externally and subsequently release the starch outside the cell wall. Several other kinds of Chlorella strains have been used such as Chlamydomonas sp. Al-5, which obtained dried algae cells of 8.5 g and Scenedesmus basilensis IAM C-66, obtained dried algae cells of 8.8 g. These cells produce starch that is less than 30% of the weight volume of the algae cell.

### EXAMPLE 2

As in Example 1, supplied air and stirring were stopped at the conclusion of the cultivation increase logarithm of the Chlorella Al-1y in a 200 liter cultivation tank containing 100 liters of the cultivation medium. A separation process of the starch from the algae cell is done by natural decantation for 16 hours. We obtained 97 g of dried starch particles by using a centrifuge and subsequently drying the starch under negative pressure at room temperature while adding acetone. The separated cultivation medium which contains the algae cells becomes 1/10 (10 liters) in volume. Another 90 liters of cultivation medium is then added to the separated medium. This process is repeated using the same processes as set forth in [0046]. After every 32 hours of cultivation, we were able to obtain starch according to the following table:

TABLE 1

| Cultivation Attempt | Obtained Starch Volume (g) |
|---|---|
| 1 | 97 |
| 2 | 94 |
| 3 | 88 |
| 4 | 91 |
| 5 | 79 |
| 6 | 83 |
| 7 | 96 |
| 8 | 87 |

The table above is representative of the ability to obtain a stable volume of starch production through the disclosed continuous cultivation process. Following the removal of the starch, 500 g of the produced starch is subjected to a saccharification process. The starch is suspended in 2.5 liters of water containing 50 ml of sulfuric acid. The suspended starch is passed through a 100 mm diameter Teflon® tube pipe at a speed of 50 mm/second. During this passage, the suspended starch is exposed to about 1.1 kilowatts and 2.45 gigahertz of microwave irradiation at a controlled temperature of about 100° to about 120° for 30 minutes. The resulting saccharified medium is tested to reveal the reduced sugar volume of the medium using the Nelson-Somogyi method. The saccharified medium is then added to a fermentation medium composed of lime milk (Ca(OH)₂) (to maintain the pH at about 6.0), 0.02% MgSO₄.7H₂O, 0.2% K₂HPO₄ and 0.1% H₂N₂CO (urea). About 500 ml of rice-based yeast (for example Saccharomyces cerevisae Kyokai No. 7, Brewing Society of Japan) is added to this fermentation medium (which contains about 20% sugar). The resulting medium is then maintained at 30° C. for 7 days until the reduced sugars become digested by the yeast.

We distilled the obtained fermentation medium (which contains ethanol) using a rotary evaporator at 50 C in a vacuum, twice. The alcohol content was 72%, measured by a float type densitometer in a total of 306 ml liquid by volume. We obtained 194 ml of more than 99% pure alcohol after processing this liquid by adding 150 g of well dried molecular sieve for 24 hours. The fermentation medium is distilled again using the same above-mentioned evaporation process. Another 20 g of dried molecular sieves is added and the medium is left for another 3 days.

### EXAMPLE 3

In Example 3, we used actual industrial wastes for cultivation. First, we cultivated a special mutant type of Chlorella vulgaris Al-1y where the mutation was induced by radiant ray irradiation (Gamma ray). The resulting algae can be cultivated faster and produce starch more efficiently and effectively. This strain can be grown under heterotrophic cultivation conditions with organic nutrients or a combined process of autotrophic and heterotrophic conditions with CO₂ gas and light irradiation, which we have previously referred to as mixotrophic cultivation.

For making this mutant algae strain, we take 2 ml of cultivated Chlorella vulgaris Al-1y and put it into a 90 mm diameter Petri dish and irradiate it by 0.4 k Gy of gamma ray irradiation. From this irradiation we achieve the desired mutation. For cultivation of this mutant algae strain, the desired mutant algae are seeded on a flat cultivation plate with an agar-agar medium containing acetic acid as the organic nutrient. Then at 35° C., the algae is supplied with a 3 times higher concentration of CO₂ gas than atmospheric air (3,000 ppm). The sample is then irradiated by using 4,000 Lux from a fluorescent lamp. The faster growing and prominent algae strain is then selected. This selected strain is implanted on a slant agar-agar plate for preservation and this plate is preserved in a 30 mm diameter test tube with an air permeable seal silicon cap for future use. When this strain is used, the cultivation process is the same as in Examples 1 and 2. This selected algae strain is planted in 8 ml of prepared cultivation medium and shook in a reciprocate shaker (shaken at a speed of 120 times/minute) for 72 hours. At the detected finishing point of the algae cultivation process, the volume of produced starch is measured using the same method as set forth in Example 1. We then proceed with either cultivation method: heterotrophic or mixotrophic.

Regarding Example 3, chemical plant wastes that contain approximately 20% (pH 2.5-4.0) of an acetic acid ion concentration were used for the carbon nutrient. The ammonium collected after the processing of methane gas from pig manure amounts to approximately 0.5% ammonium gas and was used as the nitrogen nutrient, and the carbon dioxide exhausted from oil and natural gas factories that have 10× the concentrated CO₂ gas as compared to atmospheric air were all used in these processes. We adjust the above wastes to contain 2% of acetic acid ions, 0.1% of ammonium ions and added 0.01% of MgSO₄.7H₂O, 0.008% of K₂HPO₄ and 0.002% of NaH₂PO₄. During the cultivation, air with CO₂ is supplied in the same volume as the cultivating medium, with a controlled pH of about 6 to 8 of acetic acid wastes. The above mentioned mutated Chlorella vulgaris Al-1y mentioned in [0055] is pre-cultivated by being planted in a flat glass flask 220 mm high×100 mm wide×20 mm thick with 400 ml of cultivation medium and shook for 72 hours. Thirty of these cultivation flasks are made (a total cultivation liquid volume of 12 liters) and they are controlled at 35° C., with 4,000 Lux of fluorescent light and cultivated for 6 days. We then collected the algae cells and released the starch.

Using the same analysis method set forth in Example 1, we were able to weigh out 87 g of dried algae out of the 12 liters of the cultivation medium. The starch material produced in the cell internally weighed 36 g and the starch particles that were released extra-cellularly weighed 19 g. We were able to produce alcohol through the same process of saccharification and alcohol fermentation with the use of the apparatus as mentioned in Example 2, using 10 g of produced starch particles. For all of the vapors, concentrated sulfuric acid is used to remove any unwanted water. The CO₂ gas that remains is exhausted. Since this CO₂ gas has the same mole number as the alcohol, we are able to conclude that we produced exactly 3.9 g of alcohol by weight comparison. The results concluded that about a half of the dried starch was turned to alcohol.

In addition to the above-mentioned examples of using a method of acid saccharification of starch, we are able to use another method of saccharification using biological starch saccharification agents, such as Magnax JW-101 (Rakutou Kasei Industrial Co., Ltd.; containing α-amylase and glucose amylase). In another embodiment, it is possible to simultaneously perform the processes of starch saccharification and alcohol fermentation. Yet another embodiment includes the simultaneous dual fermentation method using biological yeast such as Koji-yeast for starch saccharification and yeast-fermentation as well as using regular alcohol fermentation.

For the nitrogen nutrients, besides using ammonium produced from the processing of pig manure collected from domestic animal farmers, we are able to use any other by-products from the manure of other domestic animals from poultry farms, slaughterhouses, sewage factories or a combination of any of these materials.

In our examples, acetic acid wastes from chemical plants were used, but any other wastes such as organic acids, glycerin, any alcoholic compound or a combination of these wastes can be used as well.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention, should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations and arrangements are also possible as will be gleaned from the following claims, which are also hereby incorporated by reference into the written description. The use of terms such as “first”, “second”, “a” or “an” does not preclude the presence of additional items.

What we claim is:

1: A method of producing ethanol comprising:
cultivating starch producing unicellular green algae within a sealed tank, wherein the algae reproduce through a single cell cloning cultivation that cyclically produces starch extra cellularly;
recovering the starch, wherein the algae remain in the tank for continued cultivation;
saccharifying the starch;
fermenting the saccharified starch to produce a fermentation medium;
producing ethanol from the fermented and saccharified starch; and
recovering the ethanol produced from the fermentation medium.

2: A method according to claim 1, wherein the algae used are strains of unicellular green algae.

3: A method according to claim 1, wherein the algae are cultured at a temperature in the range of 25-42° C. and at a pH in the range of 5-9 in a closed tank.

4: A method according to claim 2, wherein the algae are cultured at a temperature in the range of 25-42° C. and at a pH in the range of 5-9 in a closed tank.

5: A method according to claim 1, wherein the algae are exposed to radiant ray or ultraviolet ray irradiation prior to starch recovery to produce more productive and selectable starch rich chlorella.

6: A method according to claim 4, wherein the algae are exposed to radiant ray or ultraviolet ray irradiation prior to starch recovery to produce more productive and selectable starch rich chlorella.

7: A method according to claim 2, wherein the Chlorella vulgaris and its induced strains are cultivated under autotrophic conditions.

8: A method according to claim 4, wherein the Chlorella vulgaris and its induced strains are cultivated under autotrophic conditions.

9: A method according to claim 6, wherein the Chlorella vulgaris and its induced strains are cultivated under autotrophic conditions.

10: A method according to claim 1, wherein the Chlorella vulgaris and its induced strains are cultivated under heterotrophic or mixotrophic conditions, wherein an aseptic sealed tank contains an assimilable nutritional cultivating medium.

11: A method according to claim 6, wherein the Chlorella vulgaris and its induced strains are cultivated under heterotrophic or mixotrophic conditions, wherein an aseptic sealed tank contains an assimilable nutritional cultivating medium.

12: A method according to claim 10, wherein the assimilable nutritional cultivating medium contains acetic acid or organic acid wastes or by-products.

13: A method according to claim 11, wherein the assimilable nutritional cultivating medium contains acetic acid or organic acid wastes or by-products.

14: A method according to claim 10, wherein the assimilable nutritional cultivating medium contains carbon dioxide gas or carbonic acid salts.

15: A method according to claim 1, wherein prior to fermentation the saccharified starch is exposed to microwave irradiation within a sealed pipeline, so that the starches are degenerated.

16: A method according to claim 4, wherein prior to fermentation the saccharified starch is exposed to microwave irradiation within a sealed pipeline, so that the starches are degenerated.

17: A method according to claim 11, wherein prior to fermentation the saccharified starch is exposed to microwave irradiation within a sealed pipeline, so that the starches are degenerated.

18: The method of claim 10, wherein the cultivation medium includes industrial waste, by-products, or combinations thereof.

19: A method of producing ethanol comprising:
cultivating Chlorella vulgaris under mixotrophic conditions within an aseptic sealed tank containing acetic acid, organic acid, carbon dioxide gas, carbonic acid salts or combinations thereof, at a temperature in the range of 25-42° C. and at a pH in the range of 5-9, wherein the algae reproduce through a single cell cloning cultivation that cyclically produces starch extra cellularly;
exposing the Chlorella vulgaris to radiant ray or ultraviolet ray irradiation;
recovering the starch, wherein the algae remain in the tank for continued cultivation;
saccharifying the starch;
exposing the saccharified starch to microwave irradiation within a sealed pipeline, so that the starches are degenerated;
fermenting the saccharified starch to produce a fermentation medium;
producing ethanol from the fermented and saccharified starch; and
recovering the ethanol produced from the fermentation medium.

20: The method of claim 19, wherein the cultivation medium includes industrial waste, by-products or combinations thereof.
